SEQUENCE LISTING (14 sequences)

SEQ ID NO: 1 - protein, 847 aa, Homo sapiens
MSLWGLVSKMPPEKVQRLYVDFPQHLRHLLGDWLESQPWEFLVGSDAFCCNLASALLSDTVQHLQASVGEQGEGSTILQHISTLESIYQRDPLKLVATFRQILQGEKKAVMEQFRHLPMPFHWKQEELKFKTGLRRLQHRVGEIHLLREALQKGAEAGQVSLHSLIETPANGTGPSEALAMLLQETTGELEAAKALVLKRIQIWKRQQQLAGNGAPFEESLAPLQERCESLVDIYSQLQQEVGAAGGELEPKTRASLTGRLDEVLRTLVTSCFLVEKQPPQVLKTQTKFQAGVRFLLGLRFLGAPAKPPLVRADMVTEKQARELSVPQGPGAGAESTGEIINNTVPLENSIPGNCCSALFKNLLLKKIKRCERKGTESVTEEKCAVLFSASFTLGPGKLPIQLQALSLPLVVIVHGNQDNNAKATILWDNAFSEMDRVPFVVAERVPWEKMCETLNLKFMAEVGTNRGLLPEHFLFLAQKIFNDNSLSMEAFQHRSVSWSQFNKEILLGRGFTFWQWFDGVLDLTKRCLRSYWSDRLIIGFISKQYVTSLLLNEPDGTFLLRFSDSEIGGITIAHVIRGQDGSPQIENIQPFSAKDLSIRSLGDRIRDLAQLKNLYPKKPKDEAFRSHYKPEQMGKDGRGYVPATIKMTVERDQPLPTPELQMPTMVPSYDLGMAPDSSMSMQLGPDMVPQVYPPHSHSIPPYQGLSPEESVNVLSAFQEPHLQMPPSLGQMSLPFDQPHPQGLLPCQPQEHAVSSPDPLLCSDVTMVEDSCLSQPVTAFPQGTWIGEDIFPPLLPPTEQDLTKLLLEGQGESGGGSLGAQPLLQPSHYGQSGISMSHMDLRANPSW

SEQ ID NO: 2 - protein, 737 aa, Homo sapiens
MEQFRHLPMPFHWKQEELKFKTGLRRLQHRVGEIHLLREALQKGAEAGQVSLHSLIETPANGTGPSEALAMLLQETTGELEAAKALVLKRIQIWKRQQQLAGNGAPFEESLAPLQERCESLVDIYSQLQQEVGAAGGELEPKTRASLTGRLDEVLRTLVTSCFLVEKQPPQVLKTQTKFQAGVRFLLGLRFLGAPAKPPLVRADMVTEKQARELSVPQGPGAGAESTGEIINNTVPLENSIPGNCCSALFKNLLLKKIKRCERKGTESVTEEKCAVLFSASFTLGPGKLPIQLQALSLPLVVIVHGNQDNNAKATILWDNAFSEMDRVPFVVAERVPWEKMCETLNLKFMAEVGTNRGLLPEHFLFLAQKIFNDNSLSMEAFQHRSVSWSQFNKEILLGRGFTFWQWFDGVLDLTKRCLRSYWSDRLIIGFISKQYVTSLLLNEPDGTFLLRFSDSEIGGITIAHVIRGQDGSPQIENIQPFSAKDLSIRSLGDRIRDLAQLKNLYPKKPKDEAFRSHYKPEQMGKDGRGYVPATIKMTVERDQPLPTPELQMPTMVPSYDLGMAPDSSMSMQLGPDMVPQVYPPHSHSIPPYQGLSPEESVNVLSAFQEPHLQMPPSLGQMSLPFDQPHPQGLLPCQPQEHAVSSPDPLLCSDVTMVEDSCLSQPVTAFPQGTWIGEDIFPPLLPPTEQDLTKLLLEGQGESGGGSLGAQPLLQPSHYGQSGISMSHMDLRANPSW

SEQ ID NO: 3 - protein, 60 aa, Mus musculus
NAKATILWDNAFSEMDRVPFVVAERVPWEKMCETLNLKFMAEVGTSRGLLPEHFLFLAQK

SEQ ID NO: 4 - protein, 59 aa, synthetic construct
NAKATILWDNAFSEMDRVPFVVAERVPWEKMCETLNLKFMAEVGTRGLLPEHFLFLAQK

SEQ ID NO: 5 - protein, 60 aa, Homo sapiens
NAKATILWDNAFSEMDRVPFVVAERVPWEKMCETLNLKFMAEVGTNRGLLPEHFLFLAQK

SEQ ID NO: 6 - protein, 60 aa, Mus musculus
IFNDNSLSVEAFQHRCVSWSQFNKEILLGRGFTFWQWFDGVLDLTKRCLRSYWSDRLIIG

SEQ ID NO: 7 - protein, 58 aa, unidentified
IFNDNSLSEAFQHRVSWSQFNKEILLGRGFTFWQWFDGVLDLTKRCLRSYWSDRLIIG

SEQ ID NO: 8 - protein, 60 aa, Homo sapiens
IFNDNSLSMEAFQHRSVSWSQFNKEILLGRGFTFWQWFDGVLDLTKRCLRSYWSDRLIIG

SEQ ID NO: 9 - protein, 60 aa, Mus musculus
FISKQYVTSLLLNEPDGTFLLRFSDSEIGGITIAHVIRGQDGSSQIENIQPFSAKDLSIR

SEQ ID NO: 10 - protein, 59 aa, synthetic construct
FISKQYVTSLLLNEPDGTFLLRFSDSEIGGITIAHVIRGQDGSQIENIQPFSAKDLSIR

SEQ ID NO: 11 - protein, 60 aa, Homo sapiens
FISKQYVTSLLLNEPDGTFLLRFSDSEIGGITIAHVIRGQDGSPQIENIQPFSAKDLSIR

SEQ ID NO: 12 - protein, 60 aa, Mus musculus
SLGDRIRDLAQLKNLYPKKPKDEAFRSHYKPEQMGKDGRGYVSTTIKMTVERDQPLPTPE

SEQ ID NO: 13 - protein, 58 aa, synthetic construct
SLGDRIRDLAQLKNLYPKKPKDEAFRSHYKPEQMGKDGRGYVTIKMTVERDQPLPTPE

SEQ ID NO: 14 - protein, 60 aa, Mus musculus
SLGDRIRDLAQLKNLYPKKPKDEAFRSHYKPEQMGKDGRGYVPATIKMTVERDQPLPTPE

(19) United States
(12) Patent Application Publication
(10) Pub. No.: US 2024/0189356 A1
(43) Pub. Date: Jun. 13, 2024

(54) ACTIVE DONOR T CELL STAT6

(71) Applicants: Mirac Nedim Ince, Iowa City, IA (US); David Elliott, Iowa City, IA (US)

(72) Inventors: Mirac Nedim Ince, Iowa City, IA (US); David Elliott, Iowa City, IA (US)

(21) Appl. No.: 18/535,619

(22) Filed: Dec. 11, 2023

Related U.S. Application Data
(60) Provisional application No. 63/386,820, filed on Dec. 9, 2022.

(51) Int. Cl. A61K 35/17; A61K 35/28; A61K 39/00; A61P 37/04; C07K 14/47; C12N 15/86

(52) U.S. Cl. CPC A61K 35/17; A61K 35/28; A61K 39/4611; A61K 39/464448; A61P 37/04; C07K 14/4705; C12N 15/86

(57) ABSTRACT

Provided herein is the use of donor T cells that express a continuously active form of STAT6, e.g., before or during BMT/HCT or after BMT/HCT, the use of autologous T cells genetically engineered to express a continuously active form of STAT6 to prevent, inhibit or treat immune diseases including autoimmune diseases and the use of T cells genetically engineered to express a continuously active form of STAT6 to enhance an immune response to a vaccine.

### CROSS-REFERENCE TO RELATED APPLICATIONS

This application claims the benefit of priority to U.S. Provisional Appl. Ser. No. 63/386,820, filed Dec. 9, 2022, which is incorporated by reference as if fully set forth herein.

### INCORPORATION BY REFERENCE OF SEQUENCE LISTING

This application contains a Sequence Listing which has been submitted electronically in ST26 format and is hereby incorporated by reference in its entirety. Said ST26 file, created on Dec. 11, 2023, is named “2392073.xml” and is 16,384 bytes in size.

### BACKGROUND

A hematopoietic stem cell transplant (HSCT; also known as a bone marrow transplant; BMT) is a procedure in which defective or cancerous bone marrow is replaced with new, healthy bone marrow cells. A stem cell transplant may be used to treat leukemia, myeloma and lymphoma, cancers that affect the blood and lymphatic system. They also can help patients recover from or better tolerate cancer treatment.

In general, the first step in stem cell transplant is harvesting healthy hematopoietic stem cells from peripheral blood or bone marrow, either from the patient or from a donor. These stem cells can then be frozen and stored. When it is ready for use, it is thawed and injected into a patient.

In an autologous stem cell or bone marrow transplant, healthy cells are harvested from peripheral blood the bone marrow of a patient. The harvested cells are frozen and stored until it is ready for use. In the meantime, the patient undergoes a ‘conditioning regimen’ to prepare their body for the transplant. In this regimen, they may receive high dose chemotherapy and/or radiation therapy. These treatments destroy cancer cells, but they also kill bone marrow cells. Autologous transplantation is most often used to treat diseases like lymphoma and multiple myeloma. Because autologous transplants use the cells of a patient, they have little to no risk of rejection or graft-versus-host disease (GVHD).

An allogeneic bone marrow or stem cell transplant uses donor stem cells to treat blood cancers that affect the bone marrow, like leukemia. The cell transplants come from a donor whose tissue most closely matches that patient. The donor cells are injected after the patient has undergone chemotherapy. Beyond restoring the blood-producing ability of the body, allogeneic stem cell transplantation can help fight cancer directly. The donated cells generate a new immune response, meaning they find and kill cancer cells, sometimes better than the original immune cells of the patient. This is called the graft-versus-tumor effect, and it can help fight cancer. Allogeneic stem cells come with an increased risk of rejection or GVHD.

Because the immune system of a patient is wiped out before a stem cell transplant, it takes about six months to a year for the immune system to recover and start producing healthy new blood cells. Transplant patients are at increased risk for infections during this time and must take precautions. Other side effects include GVHD, a condition that occurs when the body's immune cells attack cells from the donor, or when the donor cells attack your cells. GVHD can occur right after the transplant or more than a year later.

Current treatment with bone marrow transplant/hematopoietic cell transplant (BMT/HCT; also called hematopoietic stem cell transplant (HSCT)) involves the transfer of donor T cells with stem cells. Graft-versus-host disease (GVHD) is usually treated with high dose steroids. There is a need to regulate GVHD, preserve graft-versus-tumor (GVT) activity and reduce the use of toxic immune suppressive medications.

### SUMMARY

The disclosure provides for the use of donor T cells that express a continuously active form of STAT6, e.g., before or during BMT/HCT or after BMT/HCT, the use of autologous T cells genetically engineered to express a continuously active form of STAT6 to prevent, inhibit or treat immune diseases including autoimmune diseases and the use of T cells genetically engineered to express a continuously active form of STAT6 to enhance an immune response to a vaccine.

Thus, the disclosure provides for isolated human T cells genetically engineered to express a continuously (constitutive) active form of human STAT6. In one embodiment, the STAT6 has at least 86% amino acid sequence identity to SEQ ID NO:1 or SEQ ID NO:2. In one embodiment, the residue at position 548 in STAT6 is alanine, glycine, leucine or isoleucine. However, other continuously active forms of STS6 may be employed, e.g., those disclosed in Mikita et al. (J. Biol. Chem., 273:17634 (1998)), the disclosure of which is incorporated by reference herein. In one embodiment, the residues that are altered, e.g., altered to an alanine, are consecutive valine-threonine residues (VT). In one embodiment, the residues that are altered, e.g., altered to an alanine, are residues that can be phosphorylated, e.g., serine, threonine or tyrosine.

The disclosure also provides a method to enhance the efficacy of a hematopoietic cell/bone marrow transplant (HCT/BMT) in a mammal, comprising: administering to a mammal in need thereof an amount of isolated human T cells that express constitutively active STAT6. In one embodiment, the method includes administering to an amount of a population of human hematopoietic stem cells (HSCs) and an amount of isolated human T cells that express constitutively active STAT6. In one embodiment, the mammal is a human. In one embodiment, the hematopoietic stem cells are allogeneic. In one embodiment, the T cells are allogeneic. In one embodiment, the hematopoietic stem cells are autologous. In one embodiment, the T cells are autologous. In one embodiment, the STAT6 is a human STAT6. In one embodiment, the immune system of the mammal prior to administering the cells is inactivated. In one embodiment, the immune system is inactivated using chemotherapeutics or radiation. In one embodiment, the mammal has cancer. In one embodiment, the cancer is leukemia, myeloma or lymphoma. In one embodiment, the T cells comprise a viral vector expressing the STAT6.

Also provided is a method to prevent, inhibit or treat an autoimmune disease in a mammal, comprising: administering to a mammal in need thereof an amount of a composition comprising isolated human T cells that express constitutively active STAT6, a composition comprising a vector that expresses constitutively active STAT6 or a composition comprising isolated constitutively active STAT6. In one embodiment, the mammal is a human. In one embodiment, the T cells are allogeneic. In one embodiment, the T cells are autologous. In one embodiment, the STAT6 is a human STAT6. In one embodiment, the mammal has lupus or multiple sclerosis. In one embodiment, the composition comprises a viral vector expressing the STAT6. In one embodiment, the T cells are systemically administered.

In one embodiment, a method to enhance memory cell generation in a mammal is provided comprising: administering to a mammal in need thereof an amount of a composition comprising isolated T cells that express constitutively active STAT6, a composition comprising a vector that expresses constitutively active STAT6 or a composition comprising isolated constitutively active STAT6. In one embodiment, the mammal is a human. In one embodiment, the T cells are allogeneic. In one embodiment, the T cells are autologous. In one embodiment, the STAT6 is a human STAT6. In one embodiment, the composition comprises a viral vector expressing the STAT6. In one embodiment, the T cells are systemically administered. In one embodiment, memory T cells are generated by expression of, for example, STAT6VT, and those memory T cells preserve the graft versus tumor response and do not cause graft versus host disease.

In one embodiment, a method to enhance the immune response to a pathogen in a mammal is provided comprising: administering to a mammal in need thereof an amount of a composition comprising isolated T cells that express constitutively active STAT6, a composition comprising a vector that expresses constitutively active STAT6 or a composition comprising isolated constitutively active STAT6 and an immunogen of the pathogen. In one embodiment, the pathogen is a virus or a bacterium. In one embodiment, the mammal is a human. In one embodiment, the pathogen is a coronavirus. In one embodiment, the immunogen is administered before the isolated T cells or the composition. In one embodiment, the immunogen is administered after the isolated T cells or the composition. In one embodiment, the immunogen and the isolated T cells or the composition are administered concurrently.

Further provided is a population of human T cells the genome of which comprises a nucleic acid sequence encoding a constitutively active form of human STAT6. In one embodiment, the genome is augmented with an expression cassette comprising a promoter operably linked to a nucleotide sequence encoding the constitutively active form of human STAT6. In one embodiment, the promoter is a heterologous promoter. In one embodiment, the promoter comprises a CD2, CMV, stem cell virus, AAV, LTR, phosphoglycerate kinase or CAG promoter. In one embodiment, a composition is provided comprising an amount of the population of T cells and optionally a pharmaceutically acceptable carrier. In one embodiment the composition comprises naïve cell depleted T lymphocytes.

One embodiment provides a method to promote donor Tregs in a bone marrow transplant (BMT) recipient comprising administering to a mammal in need thereof an amount of isolated T cells that express constitutively active STAT6, a vector that expresses constitutively active STAT6 or isolated constitutively active STAT6.

One embodiment provides a method to preserve microbiota diversity in cecum (such as during or after HCT or BMT treatment) comprising administering to a mammal in need thereof an amount of isolated T cells that express constitutively active STAT6, a vector that expresses constitutively active STAT6 or isolated constitutively active STAT6.

Another embodiment provides a method to drive T helper 2 pathway and antitumor activity comprising administering to a mammal in need thereof an amount of isolated T cells that express constitutively active STAT6, a vector that expresses constitutively active STAT6 or isolated constitutively active STAT6.

### BRIEF DESCRIPTION OF THE FIGURES

FIG. 1A. Regulated GVHD (above) vs severe GVHD (below).

FIG. 1B. Tumor persistence due to inefficient or suboptimal GVT.

FIG. 2A. Alloreactive Lethal and Acute GVHD.

FIG. 2B. Regulation of GVHD and Preservation of GVT.

FIG. 3. STAT6VT+ T cells produce generate more T helper-2 (Th2) cytokines.

FIG. 4. STAT6VT+ T cells have increased percentage of Foxp3+ regulatory T cells in mesenteric lymph nodes (MLN) but not in the spleen.

FIG. 5. STAT6VT+ T cells display features of memory T lymphocytes

FIG. 6. BMT, GVHD induction, and GVT analysis in a mouse model.

FIG. 7. STAT6VT+ donor T cells cause a very mild GVHD.

FIGS. 8A-8F. STAT6VT+ donor T cells do not cause a severe GVHD and demonstrated a significant GVT effect. A) GVT (mouse images). B) Survival curve (‘STAT6 VT to BB survival XG, v3C’, or newer version). C) Tumor development (‘% recipients with tumor development, v2’ or newer version). D) Mortality with tumor development (‘% mortality with tumor development’ or newer version). E) Disease score (‘GVHD Scoring STAT6 paper, Disease score, TA, XG, v3’ or newer version). F) Weight loss (‘GVHD Scoring STAT6 paper, Weight loss, TA, XG, v3’ or newer version).

FIG. 9. Analysis of CD3-positive/CD4-negative/CD4-negative cells in STAT6VT+ and STAT6VT− mice. x-axis is CD4 and y-axis is CD8 cells. Gate: CD3+ lymphoid gate. The STAT6VT+ T cell population has few CD8 T cells.

FIGS. 10A-10D. STAT6VT+ transgenic splenic T lymphocytes functioned as effector memory cells after BMT in that they did not cause GVHD and promoted survival of BMT recipients (FIG. 10A-10C), but still preserved GVT reactivity (FIG. 10D). A) Survival curve (‘STAT6VT GVHD Survival, HF, XG, v2’, or newer version). B) Disease score (‘STAT6VT GVHD Disease score, HF, XG, v2’ or newer version). C) Weight loss (‘STAT6VT GVHD Weight loss, HF, XG, v3’ or newer version).

FIGS. 11A-11B. STAT6VT+ donor T cell transfer promotes the in vivo expansion of Foxp3+ and GVHD-regulating donor Tregs. Spleen and mesenteric lymph node cells were harvested from BALB/c recipient mice 6 days after transfer of T cell-depleted bone marrow (TCD-BM) cells from wild-type C57BL/6 mice and either STAT6VT− control wild-type or STAT6VT+ transgenic splenic T cells. (A) Cumulative data on Foxp3+ Treg percentage in BMT recipients of VT− (filled dots) vs VT+ donors (empty dots) from analysis of splenic T cells (left) and mesenteric lymph node (MLN) cells. *p<0.05; **p<0.01. Each dot represents one independent sample. (B) Representative examples of Foxp3+ Treg percentage from spleen (upper panels) and MLN (lower panels) cells of BMT recipient BALB/c mice of STAT6VT− control (left) or STAT6VT+ (right) donor T cells. The percentage of events in each quadrant is displayed.

FIG. 12. Transfer of STAT6VT+ T cells is associated with preservation of gut microbiota diversity after BMT. Cecal microbiota was isolated 6 days after transfer of T cell-depleted bone marrow (TCD-BM) cells from wild-type C57BL/6 mice and either STAT6VT− control wild-type or STAT6VT+ transgenic splenic T cells. 16S rRNA analysis was performed for further characterization. Data represents Shannon diversity index from two independent experiments (left (p<0.05) and right (p<0.01)) where each dot represents a microbiota sample from a single mouse.

FIG. 13. STAT6VT+ T cells are associated with a significantly different mRNA expression profile. Splenic T cells from STAT6VT+ and VT− mice were isolated before BMT and analyzed for mRNA content using bulk RNAseq. Data are derived from 3 STATVT+ and 3 STAT6VT− samples.

FIG. 14. STAT6VT+ donor T cells do not cause GVHD and demonstrated a significant GVT effect. Adding naïve T cells to STAT6VT+ T cells further increases the GVT.

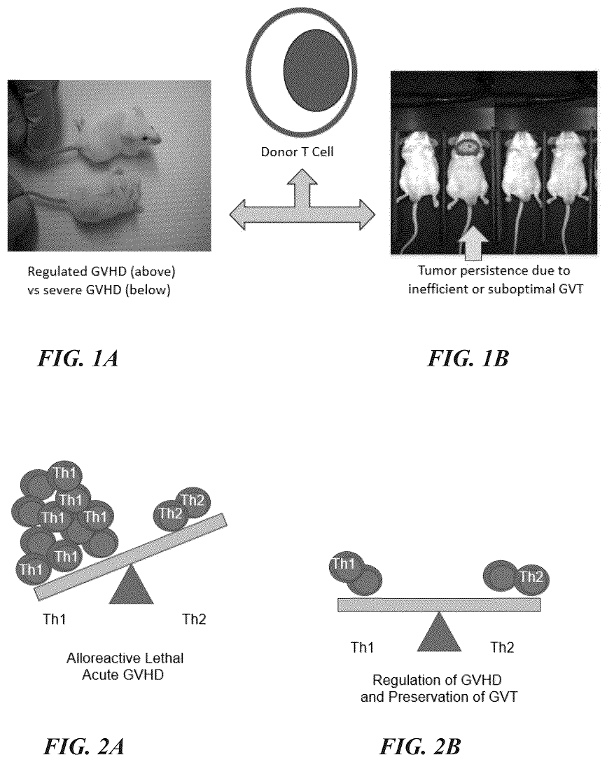

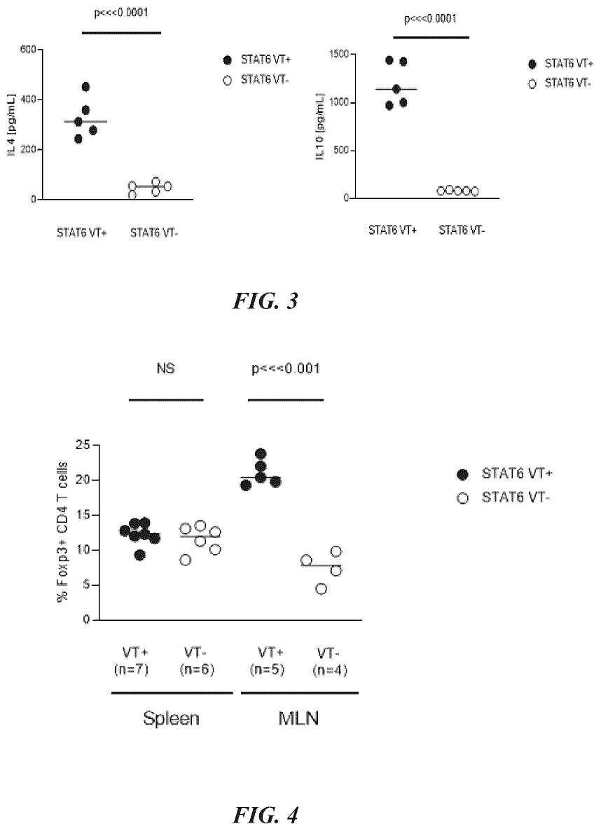

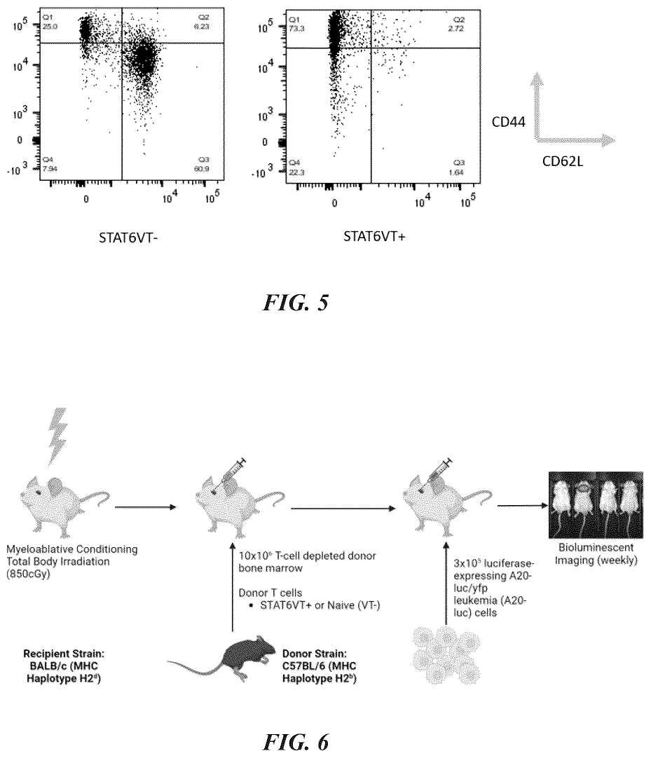

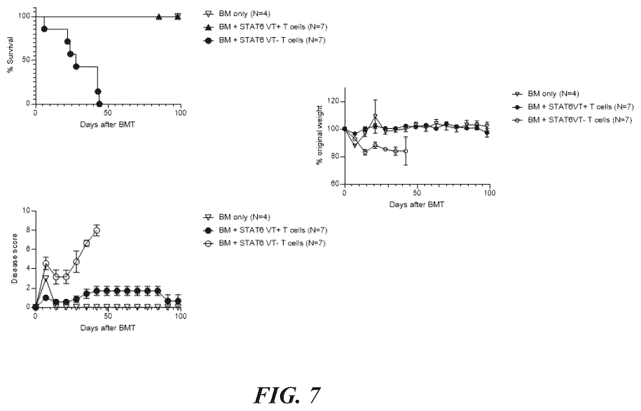

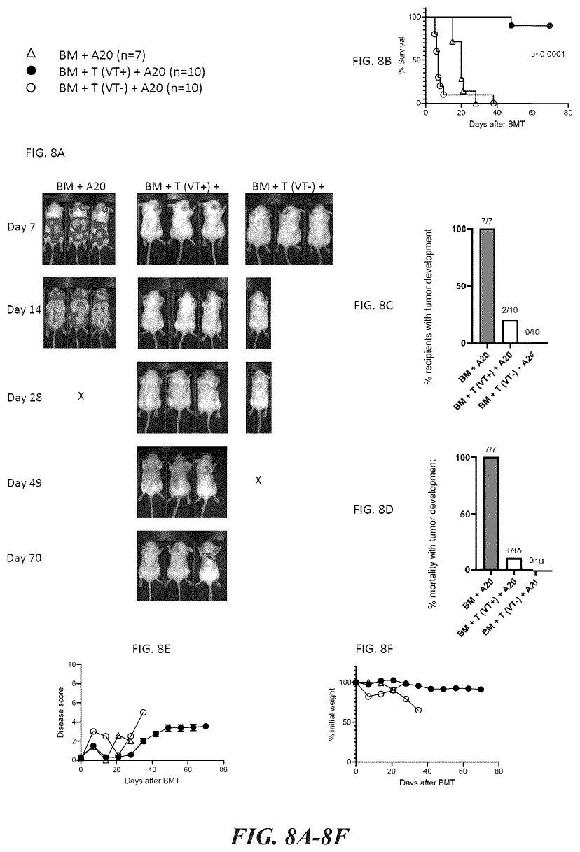

### DETAILED DESCRIPTION

Hematopoietic cell transplantation or bone marrow transplantation are curative treatment options for leukemia, myeloma, or lymphoma besides other disorders Donor T lymphocytes are transferred along with hematopoietic stem cells to prevent relapse of cancer. Unfortunately, hematopoietic cell transplantation with T lymphocytes can also cause a lethal and devastating inflammation in transplant recipients, called graft-versus-host disease (GVHD). In particular, donor T lymphocytes transferred along with bone marrow cells promote engraftment and prevent cancer relapse. The latter beneficial result of transplantation is called the graft-versus-tumor (GVT) effect. GVT relies on donor T cell reactivity against the recipient (host) tissues and as a drawback, donor T cells can cause lethal and devastating graft-versus-host disease (GVHD).

As disclosed herein, cell transplantation does not cause GVHD (harmful effect), but still prevents or retards the recurrence of cancer (the beneficial graft-versus-tumor effect), if transferred donor T lymphocytes are engineered to express a continuous active form of the protein STAT6 prior to transplantation.

STAT6 is a cellular protein that can suppress abnormal reactions by our body's defense system, which include white blood cell subsets like T lymphocytes. The disclosure provided for, in one embodiment, improvement in the outcome of hematopoietic cell transplantation if the procedure involves transfer of engineered T cells along with bone marrow cells to promote or preserve the graft-versus-tumor effect. The administration of the disclosed T cells, vectors or polypeptide likely do not cause GVHD. Furthermore, the disclosed T cells, vectors or polypeptide may be employed in other immune diseases, such as systemic lupus erythematosus including therapy-resistant systemic lupus erythematosus and other autoimmune disorders, to suppress T lymphocyte responses.

As disclosed herein, transgenic overexpression of a continuously active form of the T helper-2 (Th2) associated protein, STAT6 in T lymphocytes results in donor T cells that can preserve GVT without causing GVHD after BMT in mice. Because graft-versus-host disease is a major complication of BMT/HCT, which can negatively affect the outcome of transplantation, the disclosed methods and compositions can result in safer and widespread use BMT/HCT in patients with hematological malignancies.

### Definitions

A “vector” or “delivery” vehicle refers to a macromolecule or association of macromolecules that comprises or associates with a polynucleotide or polypeptide, and which can be used to mediate delivery of the polynucleotide or polypeptide to a cell or intercellular space, either in vitro or in vivo. Illustrative vectors include, for example, plasmids, viral vectors, liposomes, nanoparticles, or microparticles and other delivery vehicles. In one embodiment, a polynucleotide to be delivered, sometimes referred to as a “target polynucleotide” or “transgene,” may comprise a coding sequence of interest in gene therapy (such as a gene encoding a protein of therapeutic interest), a coding sequence of interest and/or a selectable or detectable marker. “Transduction,” “transfection,” “transformation” or “transducing” as used herein, are terms referring to a process for the introduction of an exogenous polynucleotide into a host cell leading to expression of the polynucleotide, e.g., the transgene in the cell, and includes the use of recombinant virus to introduce the exogenous polynucleotide to the host cell. Transduction, transfection or transformation of a polynucleotide in a cell may be determined by methods well known to the art including, but not limited to, protein expression (including steady state levels), e.g., by ELISA, flow cytometry and Western blot, measurement of DNA and RNA by hybridization assays, e.g., Northern blots, Southern blots and gel shift mobility assays. Methods used for the introduction of the exogenous polynucleotide include well-known techniques such as viral infection or transfection, lipofection, transformation and electroporation, as well as other non-viral gene delivery techniques. The introduced polynucleotide may be stably or transiently maintained in the host cell.

“Gene delivery” refers to the introduction of an exogenous polynucleotide into a cell for gene transfer, and may encompass targeting, binding, uptake, transport, localization, replicon integration and expression.

“Gene transfer” refers to the introduction of an exogenous polynucleotide into a cell which may encompass targeting, binding, uptake, transport, localization and replicon integration, but is distinct from and does not imply subsequent expression of the gene.

“Gene expression” or “expression” refers to the process of gene transcription, translation, and post-translational modification.

An “infectious” virus or viral particle is one that comprises a polynucleotide component which is capable of delivering into a cell for which the viral species is trophic. The term does not necessarily imply any replication capacity of the virus.

The term “polynucleotide” refers to a polymeric form of nucleotides of any length, including deoxyribonucleotides or ribonucleotides, or analogs thereof. A polynucleotide may comprise modified nucleotides, such as methylated or capped nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

A “transcriptional regulatory sequence” refers to a genomic region that controls the transcription of a gene or coding sequence to which it is operably linked. Transcriptional regulatory sequences of use in the present invention generally include at least one transcriptional promoter and may also include one or more enhancers and/or terminators of transcription.

“Operably linked” refers to an arrangement of two or more components, wherein the components so described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to it.

“Heterologous” means derived from a genotypically distinct entity from the entity to which it is compared. For example, a polynucleotide introduced by genetic engineering techniques into a different cell type is a heterologous polynucleotide (and, when expressed, can encode a heterologous polypeptide). Similarly, a transcriptional regulatory element such as a promoter that is removed from its native coding sequence and operably linked to a different coding sequence is a heterologous transcriptional regulatory element.

A “terminator” refers to a polynucleotide sequence that tends to diminish or prevent read-through transcription (i.e., it diminishes or prevent transcription originating on one side of the terminator from continuing through to the other side of the terminator). The degree to which transcription is disrupted is typically a function of the base sequence and/or the length of the terminator sequence. In particular, as is well known in numerous molecular biological systems, particular DNA sequences, generally referred to as “transcriptional termination sequences” are specific sequences that tend to disrupt read-through transcription by RNA polymerase, presumably by causing the RNA polymerase molecule to stop and/or disengage from the DNA being transcribed. Typical example of such sequence-specific terminators include polyadenylation (“polyA”) sequences, e.g., SV40 polyA. In addition to or in place of such sequence-specific terminators, insertions of relatively long DNA sequences between a promoter and a coding region also tend to disrupt transcription of the coding region, generally in proportion to the length of the intervening sequence. This effect presumably arises because there is always some tendency for an RNA polymerase molecule to become disengaged from the DNA being transcribed, and increasing the length of the sequence to be traversed before reaching the coding region would generally increase the likelihood that disengagement would occur before transcription of the coding region was completed or possibly even initiated. Terminators may thus prevent transcription from only one direction (“uni-directional” terminators) or from both directions (“bi-directional” terminators), and may be comprised of sequence-specific termination sequences or sequence-non-specific terminators or both. A variety of such terminator sequences are known in the art; and illustrative uses of such sequences within the context of the present invention are provided below.

“Host cells,” “cell lines,” “cell cultures,” “packaging cell line” and other such terms denote higher eukaryotic cells, such as mammalian cells including human cells, useful in the present invention, e.g., to produce recombinant virus or recombinant polypeptide. These cells include the progeny of the original cell that was transduced. It is understood that the progeny of a single cell may not necessarily be completely identical (in morphology or in genomic complement) to the original parent cell.

“Recombinant,” as applied to a polynucleotide means that the polynucleotide is the product of various combinations of cloning, restriction and/or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter. Promoters include AAV promoters, e.g., P5, P19, P40 and AAV ITR promoters, as well as heterologous promoters.

An “expression vector” is a vector comprising a region which encodes a gene product of interest, and is used for effecting the expression of the gene product in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

The terms “polypeptide” and “protein” are used interchangeably herein to refer to polymers of amino acids of any length. The terms also encompass an amino acid polymer that has been modified; for example, disulfide bond formation, glycosylation, acetylation, phosphorylation, lipidation, or conjugation with a labeling component.

An “isolated” polynucleotide, e.g., plasmid, virus, polypeptide or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Isolated nucleic acid, peptide or polypeptide is present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded). Enrichment can be measured on an absolute basis, such as weight per volume of solution, or it can be measured in relation to a second, potentially interfering substance present in the source mixture. For example, a 2-fold enrichment, 10-fold enrichment, 100-fold enrichment, or a 1000-fold enrichment.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means. An exogenous nucleic acid may be from a different organism or cell, or it may be one or more additional copies of a nucleic acid which occurs naturally within the organism or cell By way of a non-limiting example, an exogenous nucleic acid is in a chromosomal location different from that of natural cells, or is otherwise flanked by a different nucleic acid sequence than that found in nature, e.g., an expression cassette which links a promoter from one gene to an open reading frame for a gene product from a different gene.

“Transformed” or “transgenic” is used herein to include any host cell or cell line, which has been altered or augmented by the presence of at least one recombinant DNA sequence. The host cells of the present invention are typically produced by transfection with a DNA sequence in a plasmid expression vector, as an isolated linear DNA sequence, or infection with a recombinant viral vector.

The term “sequence homology” means the proportion of base matches between two nucleic acid sequences or the proportion amino acid matches between two amino acid sequences. When sequence homology is expressed as a percentage, e.g., 50%, the percentage denotes the proportion of matches over the length of a selected sequence that is compared to some other sequence. Gaps (in either of the two sequences) are permitted to maximize matching; gap lengths of 15 bases or less are usually used, 6 bases or less or 2 bases or less may be employed. When using oligonucleotides as probes or treatments, the sequence homology between the target nucleic acid and the oligonucleotide sequence is generally not less than 17 target base matches out of 20 possible oligonucleotide base pair matches (85%); not less than 9 matches out of 10 possible base pair matches (90%), or not less than 19 matches out of 20 possible base pair matches (95%).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less or 2 or less may be used. Alternatively, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. The two sequences or parts thereof are more homologous if their amino acids are greater than or equal to 50% identical when optimally aligned using the ALIGN program.

The term “corresponds to” is used herein to mean that a polynucleotide sequence is structurally related to all or a portion of a reference polynucleotide sequence, or that a polypeptide sequence is structurally related to all or a portion of a reference polypeptide sequence, e.g., they have at least 80%, 82%, 85%, 87%, 90%, 92%, 95%, 97% or more, e.g., 99% or 100%, sequence identity. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity.

The terms “substantial identity” as used herein denote a characteristic of a polynucleotide sequence, wherein the polynucleotide comprises a sequence that has at least 85 percent sequence identity, e.g., at least 90 to 95 percent sequence or at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

As used herein, “substantially pure” or “purified” means an object species is the predominant species present (i.e., on a molar basis it is more abundant than any other individual species in the composition), for instance, a substantially purified fraction is a composition wherein the object species comprises at least about 50 percent (on a molar basis) of all macromolecular species present. Generally, a substantially pure composition will comprise more than about 80 percent of all macromolecular species present in the composition, or more than about 85%, about 90%, about 95%, and about 99%. The object species may be purified to essential homogeneity (contaminant species cannot be detected in the composition by conventional detection methods) wherein the composition consists essentially of a single macromolecular species.

### Preparation of Expression Cassettes

To prepare expression cassettes encoding STAT6, variants thereof, or truncated forms thereof, for transformation, the recombinant DNA sequence or segment may be circular or linear, double-stranded or single-stranded. A DNA sequence which encodes an RNA sequence that is substantially complementary to a mRNA sequence encoding a gene product of interest is typically a “sense” DNA sequence cloned into a cassette in the opposite orientation (i.e., 3′ to 5′ rather than 5′ to 3′). Generally, the DNA sequence or segment is in the form of chimeric DNA, such as plasmid DNA, that can also contain coding regions flanked by control sequences which promote the expression of the DNA in a cell. As used herein, “chimeric” means that a vector comprises DNA from at least two different species, or comprises DNA from the same species, which is linked or associated in a manner which does not occur in the “native” or wild-type of the species.

Aside from DNA sequences that serve as transcription units, or portions thereof, a portion of the DNA may be untranscribed, serving a regulatory or a structural function. For example, the DNA may itself comprise a promoter that is active in eukaryotic cells, e.g., mammalian cells, or in certain cell types, or may utilize a promoter already present in the genome that is the transformation target of the lymphotropic virus. Such promoters include the CMV promoter, as well as the SV40 late promoter and retroviral LTRs (long terminal repeat elements), although many other promoter elements well known to the art may be employed, e.g., the MMTV, RSV, MLV or HIV LTR in the practice of the invention. In one embodiment, expression is inducible. In one embodiment, a tissue-specific promoter (or enhancer) is employed. In one embodiment, a T cell specific promoter is employed.

Other elements functional in the host cells, such as introns, enhancers, polyadenylation sequences and the like, may also be a part of the recombinant DNA. Such elements may or may not be necessary for the function of the DNA, but may provide improved expression of the DNA by affecting transcription, stability of the mRNA, or the like. Such elements may be included in the DNA as desired to obtain the optimal performance of the transforming DNA in the cell.

The recombinant DNA to be introduced into the cells may contain either a selectable marker gene or a reporter gene or both to facilitate identification and selection of transformed cells from the population of cells sought to be transformed. Alternatively, the selectable marker may be carried on a separate piece of DNA and used in a co-transformation procedure. Both selectable markers and reporter genes may be flanked with appropriate regulatory sequences to enable expression in the host cells. Useful selectable markers are well known in the art and include, for example, antibiotic and herbicide-resistance genes, such as neo, hpt, dhfr, bar, aroA, puro, hyg, dapA and the like. See also, the genes listed on Table 1 of Lundquist et al. (U.S. Pat. No. 5,848,956).

Reporter genes are used for identifying potentially transformed cells and for evaluating the functionality of regulatory sequences. Reporter genes which encode for easily assayable proteins are well known in the art. In general, a reporter gene is a gene which is not present in or expressed by the recipient organism or tissue and which encodes a protein whose expression is manifested by some easily detectable property, e.g., enzymatic activity. Exemplary reporter genes include the chloramphenicol acetyl transferase gene (cat) from Tn9 of E. coli, the beta-glucuronidase gene (gus) of the uidA locus of E. coli, the green, red, or blue fluorescent protein gene, and the luciferase gene. Expression of the reporter gene is assayed at a suitable time after the DNA has been introduced into the recipient cells. A selectable marker gene, e.g., for either positive or negative selection, may also be employed

The general methods for constructing recombinant DNA which can transform target cells are well known to those skilled in the art, and the same compositions and methods of construction may be utilized to produce the DNA useful herein.

The recombinant DNA can be readily introduced into the host cells, e.g., mammalian, bacterial, yeast or insect cells, or prokaryotic cells, by transfection with an expression vector comprising the recombinant DNA by any procedure useful for the introduction into a particular cell, e.g., physical or biological methods, to yield a transformed (transgenic) cell having the recombinant DNA so that the DNA sequence of interest is expressed by the host cell. In one embodiment, the recombinant DNA is stably integrated into the genome of the cell.

Physical methods to introduce a recombinant DNA into a host cell include calcium-mediated methods, lipofection, particle bombardment, microinjection, electroporation, and the like. Biological methods to introduce the DNA of interest into a host cell include the use of DNA and RNA viral vectors. Viral vectors, e.g., retroviral or lentiviral vectors, have become a widely used method for inserting genes into eukaryotic cells, such as mammalian, e.g., human cells. Other viral vectors can be derived from poxviruses, e.g., vaccinia viruses, herpes viruses, adenoviruses, adeno-associated viruses, baculoviruses, and the like.

To confirm the presence of the recombinant DNA sequence in the host cell, a variety of assays may be performed. Such assays include, for example, molecular biological assays well known to those of skill in the art, such as Southern and Northern blotting, RT-PCR and PCR; biochemical assays, such as detecting the presence or absence of a particular gene product, e.g., by immunological means (ELISAs and Western blots) or by other molecular assays.

To detect and quantitate RNA produced from introduced recombinant DNA segments, quantitative RT-PCR may be employed. In this application of PCR, RNA is reverse transcribed into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique demonstrates the presence of an RNA species and gives information about the integrity of that RNA. The presence or absence of an RNA species can also be determined using dot or slot blot Northern hybridizations. These techniques are modifications of Northern blotting and only demonstrate the presence or absence of an RNA species.

While Southern blotting and PCR may be used to detect the recombinant DNA segment in question, they do not provide information as to whether the recombinant DNA segment is being expressed. Expression may be evaluated by specifically identifying the peptide products of the introduced DNA sequences or evaluating the phenotypic changes brought about by the expression of the introduced DNA segment in the host cell.

### Vectors for Delivery

Delivery vectors include, for example, nucleic acid vectors, e.g., plasmids, viral vectors, and isolated linear nucleic acid, either single-stranded or double stranded, microparticles, nanoparticles, liposomes and other lipid-containing complexes, and other macromolecular complexes capable of mediating delivery of a gene, polypeptide or transgenic cells to a host cell, e.g., to provide for recombinant expression of a polypeptide encoded by the gene. Vectors can also comprise other components or functionalities that further modulate gene delivery and/or gene expression, or that otherwise provide beneficial properties. Such other components include, for example, components that influence binding or targeting to cells (including components that mediate cell-type or tissue-specific binding); components that influence uptake of the vector by the cell; components that influence localization of the transferred gene within the cell after uptake (such as agents mediating nuclear localization); and components that influence expression of the gene. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e., positive/negative) markers (see, e.g., WO 92/08796; and WO 94/28143). Such marker genes can provide an added measure of control that can be advantageous in gene therapy contexts. A large variety of such vectors are known in the art and are generally available.

Vectors within the scope of the disclosure include, but are not limited to, isolated nucleic acid, e.g., plasmid-based vectors which may be extra-chromosomally maintained, and viral vectors, e.g., recombinant adenovirus, retrovirus, lentivirus, herpesvirus, poxvirus, papilloma virus, or adeno-associated virus, including viral and non-viral vectors which are present in liposomes, e.g., neutral or cationic liposomes, such as DOSPA/DOPE, DOGS/DOPE or DMRIE/DOPE liposomes, and/or associated with other molecules such as DNA-anti-DNA antibody-cationic lipid (DOTMA/DOPE) complexes. Vectors may be administered in vivo via any route including, but not limited to, intramuscular, buccal, rectal, intravenous or oral administration, and transfer to cells, e.g., in vitro, may be enhanced using electroporation and/or iontophoresis. In one embodiment, vectors are locally administered.

In one embodiment, an isolated polynucleotide or vector having that polynucleotide comprises nucleic acid encoding a polypeptide or fusion protein that has substantial identity, e.g., at least 80% or more, e.g., 85%, 87%, 90%, 92%, 95%, 97%, 98%, 99% and up to 100%, amino acid sequence identity to one of SEQ ID NOs: 1-2.

### Polypeptides and Fusion Proteins

The polypeptide or fusion proteins can be synthesized in vitro, e.g., by the solid phase peptide synthetic method or by recombinant DNA approaches (see above). The solid phase peptide synthetic method is an established and widely used method. These polypeptides can be further purified by fractionation on immunoaffinity or ion-exchange columns; ethanol precipitation; reverse phase HPLC; chromatography on silica or on an anion-exchange resin such as DEAE; chromatofocusing; SDS-PAGE; ammonium sulfate precipitation, gel filtration using, for example, Sephadex G-75; or ligand affinity chromatography.

Once isolated and characterized, chemically modified derivatives of a given polypeptide or fusion thereof, can be readily prepared. For example, amides of the polypeptide or fusion thereof of the present invention may also be prepared by techniques well known in the art for converting a carboxylic acid group or precursor, to an amide. One method for amide formation at the C-terminal carboxyl group is to cleave the polypeptide or fusion thereof from a solid support with an appropriate amine, or to cleave in the presence of an alcohol, yielding an ester, followed by aminolysis with the desired amine.

Salts of carboxyl groups of a polypeptide or fusion thereof may be prepared in the usual manner by contacting the polypeptide, or fusion thereof with one or more equivalents of a desired base such as, for example, a metallic hydroxide base, e.g., sodium hydroxide; a metal carbonate or bicarbonate base such as, for example, sodium carbonate or sodium bicarbonate; or an amine base such as, for example, triethylamine, triethanolamine, and the like.

N-acyl derivatives of an amino group of the polypeptide or fusion thereof may be prepared by utilizing an N-acyl protected amino acid for the final condensation, or by acylating a protected or unprotected peptide, polypeptide, or fusion thereof. O-acyl derivatives may be prepared, for example, by acylation of a free hydroxy polypeptide or polypeptide resin. Either acylation may be carried out using standard acylating reagents such as acyl halides, anhydrides, acyl imidazoles, and the like. Both N- and O-acylation may be carried out together, if desired.

Formyl-methionine, pyroglutamine and trimethyl-alanine may be substituted at the N-terminal residue of the polypeptide. Other amino-terminal modifications include aminooxypentane modifications.

In one embodiment, a polypeptide or fusion protein has substantial identity, e.g., at least 80% or more, e.g., 85%, 87%, 90%, 92%, 95%, 97%, 98%, 99% and up to 100%, amino acid sequence identity to one of SEQ ID NOs: 1-2.

Substitutions may include substitutions which utilize the D rather than L form, as well as other well-known amino acid analogs, e.g., unnatural amino acids such as α, α-disubstituted amino acids, N-alkyl amino acids, lactic acid, and the like. These analogs include phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoyl-phenylalanine, phenylglycine, propargylglycine, sarcosine, ε-N,N,N-trimethyllysine, ε-N-acetyllysine, N-acetylserine, N-formylmethionine, 3-methylhistidine, 5-hydroxylysine, ω-N-methylarginine, and other similar amino acids and imino acids and tert-butylglycine.

Conservative amino acid substitutions may be employed—that is, for example, aspartic-glutamic as acidic amino acids; lysine/arginine/histidine as polar basic amino acids; leucine/isoleucine/methionine/valine/alanine/proline/glycine non-polar or hydrophobic amino acids; serine/threonine as polar or hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting peptide, polypeptide or fusion polypeptide. Whether an amino acid change results in a functional peptide, polypeptide or fusion polypeptide can readily be determined by assaying the specific activity of the peptide, polypeptide or fusion polypeptide.

Amino acid substitutions falling within the scope of the disclosure, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic; trp, tyr, phe.

The invention also envisions a polypeptide or fusion polypeptide with non-conservative substitutions. Non-conservative substitutions entail exchanging a member of one of the classes described above for another.

Acid addition salts of the polypeptide or fusion polypeptide or of amino residues of the polypeptide or fusion polypeptide may be prepared by contacting the polypeptide or amine with one or more equivalents of the desired inorganic or organic acid, such as, for example, hydrochloric acid. Esters of carboxyl groups of the polypeptides may also be prepared by any of the usual methods known in the art.

### Formulations and Dosages

The T cells, polypeptides or fusions thereof, or nucleic acid encoding the polypeptide or fusion, can be formulated as pharmaceutical compositions and administered to a mammalian host, such as a human patient in a variety of forms adapted to the chosen route of administration, e.g., orally or parenterally, by intravenous, intramuscular, topical or subcutaneous routes. In one embodiment, the polypeptide or nucleic acid encoding the polypeptide is systemically administered or is administered prophylactically.

In one embodiment, the T cells, polypeptides or fusions thereof, or nucleic acid encoding the polypeptide or fusion, may be administered by infusion or injection. Solutions of the T cells, polypeptides or fusions thereof, or nucleic acid encoding the polypeptide or fusion or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion may include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it may be desirable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active agent in the required amount in the appropriate solvent with various of the other ingredients enumerated above, as required, followed by filter sterilization. In the case of sterile powders for the preparation of sterile injectable solutions, the methods of preparation include vacuum drying and the freeze drying techniques, which yield a powder of the active ingredient plus any additional desired ingredient present in the previously sterile-filtered solutions.

Useful solid carriers may include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as antimicrobial agents can be added to optimize the properties for a given use. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the T cells, polypeptides or fusions thereof, or nucleic acid encoding the polypeptide or fusion, can be determined by comparing their in vitro activity and in vivo activity in animal models thereof. Methods for the extrapolation of effective dosages in mice, and other animals, to humans are known to the art; for example, see U.S. Pat. No. 4,938,949.

Generally, the concentration of the T cells, polypeptides or fusions thereof, or nucleic acid encoding the polypeptide or fusion, in a liquid composition, may be from about 0.1-25 wt-%, e.g., from about 0.5-10 wt-%. The concentration in a semi-solid or solid composition such as a gel or a powder may be about 0.1-5 wt-%, e.g., about 0.5-2.5 wt-%.

The amount of the T cells, polypeptides or fusions thereof, or nucleic acid encoding the polypeptide or fusion for use alone or with other agents will vary with the route of administration, the nature of the condition being treated and the age and condition of the patient and will be ultimately at the discretion of the attendant physician or clinician.

The T cells, polypeptides or fusions thereof, or nucleic acid encoding the polypeptide or fusion, may be conveniently administered in unit dosage form; for example, containing 5 to 1000 mg, conveniently 10 to 750 mg, or conveniently 50 to 500 mg of active ingredient per unit dosage form.

In general, however, a suitable dose, e.g., of the polypeptide or isolated nucleic acid, may be in the range of from about 0.5 to about 100 mg/kg, e.g., from about 10 to about 75 mg/kg of body weight per day, such as 3 to about 50 mg per kilogram body weight of the recipient per day, for example in the range of 6 to 90 mg/kg/day, e.g., in the range of 15 to 60 mg/kg/day.

For instance, the polypeptides or fusions thereof, or nucleic acid encoding the polypeptide or fusion, may be conveniently administered in unit dosage form; for example, containing 5 to 1000 mg, conveniently 10 to 750 mg, or conveniently 50 to 500 mg of active ingredient per unit dosage form.

For HSC transplant, a suitable dose of, for example. CD34+ cells, may be from 0.5×10⁶ to about 1×10⁹CD34+/kg, e.g., from 0.75×10⁶ to about 1×10⁷CD34+/kg, from 1.0×10⁶ to about 1×10⁷ CD34+/kg, or from 5×10⁶ to about 1×10⁸CD34+/kg.

For T cell transplant, a suitable dose may be from 0.5×10⁷ to about 1×10¹⁰ T cells, e.g., from 0.75×10⁷ to about 1×10⁹ T cells, from 1.0×10⁷ to about 1×10⁸ T cells, or from 5×10⁷ to about 5×10⁸ T cells

### Exemplary STAT6 Sequences

The STAT6 employed in the cells, encoded by the vectors and/or employed as isolated polypeptide may have a sequence in NCBI Reference Sequence. NP_003144.3:

(SEQ ID NO: 1)
MSLWGLVSKM PPEKVQRLYV DFPQHLRHLL GDWLESQPWE
FLVGSDAFCC NLASALLSDT VQHLQASVGE QGEGSTILQH
ISTLESIYQR DPLKLVATFR QILQGEKKAV MEQFRHLPMP
FHWKQEELKF KTGLRRLQHR VGEIHLLREA LQKGAEAGQV
SLHSLIETPA NGTGPSEALA MLLQETTGEL EAAKALVLKR
IQIWKRQQQL AGNGAPFEES LAPLQERCES LVDIYSQLQQ
EVGAAGGELE PKTRASLTGR LDEVERTIVT SCFLVEKQPP
QVLKTQTKFQ AGVRFLLGLR FLGAPAKPPL VRADMVTEKQ
ARELSVPQGP GAGAESTGEI INNTVPLENS IPGNCCSALF
KNLLLKKIKR CERKGTESVT EEKCAVLFSA SFTLGPGKLP
IQLQALSLPL VVIVHGNQDN NAKATILWDN AFSEMDRVPF
VVAERVPWEK MCETLNLKFM AEVGTNRGLL PEHFLFLAQK
IFNDNSLSME AFQHRSVSWS QFNKEILLGR GFTFWQWFDG
VLDLTKRCLR SYWSDRLIIG FISKQYVTSL LLNEPDGTFL
LRFSDSEIGG ITIAHVIRGQ DGSPQIENIQ PFSAKDLSIR
SLGDRIRDLA QLKNLYPKKP KDEAFRSHYK PEQMGKDGRG
YVPATIKMTV ERDQPLPTPE LQMPTMVPSY DLGMAPDSSM
SMQLGPDMVP QVYPPHSHSI PPYQGLSPEE SVNVLSAFQE
PHLQMPPSLG QMSLPFDQPH PQGLLPCQPQ EHAVSSPDPL
LCSDVTMVED SCLSQPVTAF PQGTWIGEDI FPPLLPPTEQ
DLTKLLLEGQ GESGGGSLGA QPLLQPSHYG QSGISMSHMD
LRANPSW

### NCBI Reference Sequence: NP_001171551.1

(SEQ ID NO: 2)
MEQFRHLPMP FHWKQEELKF KTGLRRLQHR VGEIHLLREA
LQKGAEAGQV SLHSLIETPA NGTGPSEALA MLLQETTGEL
EAAKALVLKR IQIWKRQQQL AGNGAPFEES LAPLQERCES
LVDIYSQLQQ EVGAAGGELE PKTRASLTGR LDEVLRTLVT
SCFLVEKQPP QVLKTQTKFQ AGVRFLLGLR FLGAPAKPPL
VRADMVTEKQ ARELSVPQGP GAGAESTGEI INNTVPLENS
IPGNCCSALF KNLLLKKIKR CERKGTESVT EEKCAVLFSA
SFTLGPGKLP IQLQALSLPL VVIVHGNQDN NAKATILWDN
AFSEMDRVPF VVAERVPWEK MCETLNLKFM AEVGTNRGLL
PEHFLFLAQK IFNDNSLSME AFQHRSVSWS QFNKEILLGR
GFTFWQWFDG VLDLTKRCLR SYWSDRLIIG FISKQYVTSL
LLNEPDGTFL LRFSDSEIGG ITIAHVIRGQ DGSPQIENIQ
PFSAKDLSIR SLGDRIRDLA QLKNLYPKKP KDEAFRSHYK
PEQMGKDGRG YVPATIKMTV ERDQPLPTPE LQMPTMVPSY
DLGMAPDSSM SMQLGPDMVP QVYPPHSHSI PPYQGLSPEE
SVNVLSAFQE PHLQMPPSLG QMSLPFDQPH PQGLLPCQPQ
EHAVSSPDPL LCSDVTMVED SCLSQPVTAF PQGTWIGEDI
FPPLLPPTEQ DLTKLLLEGQ GESGGGSLGA QPLLQPSHYG
QSGISMSHMD LRANPSW

or a polypeptide having at least 80%, e.g., 86%, 87%, 88%, 89% 90%, 92%, 95%, 97%, 98% or 99%, including any integer between 80 and 99, contiguous amino acid sequence identity thereto. In one embodiment, the polypeptide has a residue other than V at position 547, a residue other than T at position 548, or a residue other than V at position 547 and a residue other than T at position 548. In one embodiment, the residue at position 547, 548 or both, is a residue that is not capable of being phosphorylated.

The alignment of the C-terminus of wild-type mouse and human STAT6 is shown below with the residues noted above highlighted:

Sequence A
(SEQ ID NO: 3)
NAKATILWDNAFSEMDRVPFVVAERVPWEKMCETLNLKFMAEVGTSRGLL
PEHFLFLAQK
Aligned
(SEQ ID NO: 4)
NAKATILWDNAFSEMDRVPFVVAERVPWEKMCETLNLKFMAEVGT +
RGLLPEHFLFLAQK
Sequence B
(SEQ ID NO: 5)
NAKATILWDNAFSEMDRVPFVVAERVPWEKMCETINLKFMAEVGTNRGLL
PEHFLFLAQK
Sequence A
(SEQ ID NO: 6)
IFNDNSLSVEAFQHRCVSWSQFNKEILLGRGFTFWQWFDGVLDLTKRCLR
SYWSDRLIIG
Aligned
(SEQ ID NO: 7)
IFNDNSLS + EAFQHRVSWSQFNKEILLGRGFTFWQWFDGVLDLTKRCL
RSYWSDRLIIG
Sequence B
(SEQ ID NO: 8)
IFNDNSLSMEAFQHRSVSWSQFNKEILLGRGFTFWQWFDGVLDLTKRCLR
SYWSDRLIIG
Sequence A
(SEQ ID NO: 9)
FISKQY [symbol] SLLLNEPDGTFLLRFSDSEIGGITIAHVIRGQDGSQIENIQP
FSAKDLSIR
Aligned
(SEQ ID NO: 10)
FISKQY [symbol] SLLLNEPDGTFLLRFSDSEIGGITIAHVIRGQDGSQIENIQP
FSAKDLSIR
Sequence B
(SEQ ID NO: 11)
FISKQY [symbol] SLLLNEPDGTFLLRFSDSEIGGITIAHVIRGQDGSPQIENIQ
PFSAKDLSIR
Sequence A
(SEQ ID NO: 12)
SLGDRIRDLAQLKNLYPKKPKDEAFRSHYKPEQMGKDGRGYVSTTIKMTV
ERDQPLPTPE
Aligned
(SEQ ID NO: 13)
SLGDRIRDLAQLKNLYPKKPKDEAFRSHYKPEQMGKDGRGYV
TIKMTVERDQPLPTPE
Sequence B
(SEQ ID NO: 14)
SLGDRIRDLAQLKNLYPKKPKDEAFRSHYKPEQMGKDGRGYVPATIKMTV
ERDQPLPTPE

### Exemplary Diseases

In one embodiment, the T cells expressing continuously active STAT6, isolated STAT6 polypeptide or nucleic acid vector encoding STAT6, may be administered to a mammal having or suspected of having a pathogen infection, or a mammal being administered a vaccine for a pathogen. In one embodiment, the pathogen is a virus. In one embodiment, the pathogen is bacterium. In one embodiment, the pathogen is a parasite. In one embodiment, the virus is a coronavirus. In one embodiment, the virus is an influenza virus. In one embodiment, the virus is a respiratory syncytial virus. In one embodiment, the bacterium is pneumococcus. In one embodiment, the bacterium is meningococcus. In one embodiment, the virus is a morbillivirus, e.g., causing measles. In one embodiment, the virus is a mumps virus. In one embodiment, the pathogen is Plasmodium. In one embodiment, the pathogen causes Rubella. In one embodiment, the pathogen is Clostridium, e.g., one causing tetanus. In one embodiment, the pathogen is Bordetella, e.g., one causing pertussis. In one embodiment, the pathogen is Salmonella, e.g., one causing typhoid. In one embodiment, the pathogen is a variola virus, e.g., one causing smallpox. In one embodiment, the pathogen is Corynebacterium, e.g., one causing diphtheria. In one embodiment, the pathogen is human papilloma virus. In one embodiment, the pathogen is HIV. In one embodiment, the pathogen is a hepatitis virus, e.g., HCV or HEV.

In one embodiment, the T cells expressing recombinant STAT6, isolated STAT6polypeptide or nucleic acid vector encoding STAT6, may be administered to a mammal having or suspected of having an autoimmune disease, e.g., to alter one or more symptoms of the disease. Thus, the compositions described herein may be employed to prevent, inhibit or treat an autoimmune disease Autoimmune diseases include but are not limited to Addison disease, Celiac disease-sprue (gluten-sensitive enteropathy), Dermatomyositis, Graves' disease, Hashimoto thyroiditis, Multiple sclerosis, Myasthenia gravis, Pernicious anemia, Reactive arthritis, Rheumatoid arthritis, Sjögren syndrome, Systemic lupus erythematosus, or Type I diabetes. In one embodiment, the autoimmune disease is type I diabetes. In type 1 diabetes mellitus, the immune system attacks and destroys insulin-producing cells in the pancreas. High blood sugar results can damage the blood vessels and organs, including the heart, kidneys, eyes, and nerves. In one embodiment, the autoimmune disease is rheumatoid arthritis. In rheumatoid arthritis (RA), the immune system attacks the joints. This attack causes redness, warmth, soreness, and stiffness in the joints. In one embodiment, the autoimmune disease is psoriasis/psoriatic arthritis. Psoriasis causes skin cells to multiply too quickly. The extra cells build up and form inflamed, red patches, commonly with silver-white scales of plaque on lighter-toned skin. On darker skin, psoriasis can appear purplish or dark brown with gray scales. Up to 30% of people with psoriasis also develop swelling, stiffness, and pain in their joints. This form of the disease is called psoriatic arthritis. In one embodiment, the autoimmune disease is multiple sclerosis. Multiple sclerosis (MS) damages the myelin sheath, the protective coating surrounding nerve cells in your central nervous system. Damage to the myelin sheath slows the transmission speed of messages between your brain and spinal cord to and from the rest of your body. This damage can lead to numbness, weakness, balance issues, and trouble walking.

In one embodiment, the autoimmune disease is systemic lupus erythematosus. The systemic form, which is most common, actually affects many organs, including the joints, kidneys, brain, and heart. Joint pain, fatigue, and rashes are among the most common symptoms In one embodiment, the autoimmune disease is inflammatory bowel disease. Inflammatory bowel disease (IBD) describes conditions that cause inflammation in the lining of the intestinal wall. Each type of IBD affects a different part of the GI tract. Crohn's disease can inflame any part of the GI tract, from the mouth to the anus. Ulcerative colitis affects only the lining of the large intestine (colon) and rectum. In one embodiment, the autoimmune disease is Addison's disease. Addison's disease affects the adrenal glands, which produce the hormones cortisol and aldosterone as well as androgen hormones. Too little cortisol can affect how the body uses and stores carbohydrates and sugar (glucose). Deficiency of aldosterone will lead to sodium loss and excess potassium in the bloodstream. Symptoms include weakness, fatigue, weight loss, and low blood sugar. In one embodiment, the autoimmune disease is Graves' disease. Graves' disease attacks the thyroid gland in the neck, causing it to produce too much of its hormones. Thyroid hormones control the body's energy usage, known as metabolism. Having too much of these hormones causes symptoms like nervousness, a fast heartbeat, heat intolerance, and weight loss. One potential symptom of this disease is bulging eyes, called exophthalmos. In one embodiment, the autoimmune disease is Sjogren's syndrome. This condition attacks the glands that provide lubrication to the eyes and mouth. The hallmark symptoms of Sjögren's syndrome are dry eyes and dry mouth, but it may also affect the joints or skin. In one embodiment, the autoimmune disease is Hashimoto's thyroiditis. In Hashimoto's thyroiditis, thyroid hormone production slows to a deficiency. Symptoms include weight gain, sensitivity to cold, fatigue, hair loss, and swelling of the thyroid (goiter). In one embodiment, the autoimmune disease is Myasthenia gravis. Myasthenia gravis affects nerve impulses that help the brain control the muscles. When the communication from nerves to muscles is impaired, signals can't direct the muscles to contract. The most common symptom is muscle weakness, which worsens with activity and improves with rest. Muscles that control eye movements, eyelid opening, swallowing, and facial movements are often involved. In one embodiment, the autoimmune disease is autoimmune vasculitis. Autoimmune vasculitis happens when the immune system attacks blood vessels. The inflammation that results narrows the arteries and veins, allowing less blood to flow through them. In one embodiment, the autoimmune disease is pernicious anemia. This condition causes a deficiency of a protein made by stomach lining cells, which is an intrinsic factor needed for the small intestine to absorb vitamin B12 from food. Without enough of this vitamin, one will develop anemia, and the body's ability for proper DNA synthesis will be altered In one embodiment, the autoimmune disease is celiac disease. People with celiac disease can't eat foods containing gluten, a protein found in wheat, rye, and other grain products. When gluten is in the small intestine, the immune system attacks this part of the gastrointestinal tract and causes inflammation.

The T cells expressing STAT6 may be employed in mammals having cancer, for example, B cell cancers. B cell cancers include B-cell lymphoma. Types of B cell lymphoma include but are not limited to diffuse large B-cell lymphoma, follicular lymphoma, small lymphocytic lymphoma (SLL)/Chronic lymphocytic leukemia (CLL), Mantle cell lymphoma (MCL), Marginal zone lymphoma, and Burkitt lymphoma. Diffuse large B-cell lymphoma (DLBCL) is the most common type of B-cell lymphoma. DLBCL typically affects older people and accounts for one out of three cases of non-Hodgkin lymphoma. This type of B-cell lymphoma usually begins as a mass in a lymph node, but can also form in particular sites such as bone, intestine, the spinal cord or brain. Standard treatment for DLBCL generally involves months of conventional chemotherapy, and overall cure rates in the last 15 years have been about 70%.

Diffuse large B-cell lymphoma has long been treated as a single disease, but it is now categorized into many different subtypes based on their molecular features. There are two large subtypes: germinal center b-cell (GCB) and activated b-cell (ABC).

Diffuse large B-cell lymphoma can also be categorized based on where it develops. Site-specific forms include: Primary mediastinal B-cell lymphoma (located within the chest, behind the sternum) has distinctive clinical and molecular features. It is significantly different than other types of DLBCL and, biologically, bas more in common with classic Hodgkin lymphoma. Primary central nervous system lymphoma is rare in general, but is more likely to develop in older populations, as well as those with compromised immune systems. It is almost always DLBCL, particularly the ABC subtype. It can sometimes present in tissues surrounding the eye or spinal cord, but is mostly found throughout the brain and spinal cord.

Other diseases that are amenable to treatment with the T cells, polypeptide or nucleic acid include Waldenstrom macroglobulinemia, and leukemias including Hairy cell leukemia. There are 4 main types of leukemia, based on whether they are acute or chronic, and myeloid or lymphocytic:acute myeloid (or myelogenous) leukemia (AML), chronic myeloid (or myelogenous) leukemia (CML), acute lymphocytic (or lymphoblastic) leukemia (ALL), and chronic lymphocytic leukemia (CLL).

The invention will be further described by the following non-limiting examples.

### Example 1

Stimulation of T helper-2 (Th2) pathway regulates acute graft-versus-host disease (GVHD) and preserves the graft-versus-tumor (GVT) effect after bone marrow transplantation (BMT). Cellular proteins associated with Th2 maturation include those induced by the transcription factor STAT6, which promotes immune regulation and alleviates GVHD in different models of BMT. In this study, it was investigated whether augmentation of the Th2 pathway in donor T cells by genetic overexpression of STAT6 regulates GVHD and preserves GVT activity.

A BMT and acute GVHD model with major MHC I/II mismatch (H2b into H2d) was employed after myeloablative preparation with total body irradiation (850 cGy). Donor T cells from STAT6VT+ transgenic mice were used on the H2b background whose T cells overexpress a constitutively active form of STAT6, driven by the CD2 promoter. STAT6VT− nontransgenic mice (H2b) were also used as donor T cell source and C57BL/6 wildtype (WT)(H2b) mice to provide donor T cell-depleted bone marrow (TCD-BM) cells as well as splenic donor T lymphocytes. WT BALB/c mice (H2d) constituted BMT recipients. Luciferase expressing A20 leukemia/lymphoma cell line syngeneic with BALB/c recipients (H2d) was used for graft-versus-tumor (GVT) studies. Tumor load assessment in BMT recipients was performed using bioluminescence imaging.

T cells from STAT6VT+ transgenic mice generated high quantities of Th2 and immune regulatory cytokines, such as interleukin-4 (VT+: 2.8±0.2 ng/ml vs. VT−: 0.1±0.03 ng/ml; p<0.0001) or interleukin-10 (VT+: 700±89 pg/ml vs. VT−: 107±10 pg/ml, p<0.0001)(Data are representative examples from at least 4 independent experiments for each cytokine and calculated as meant SD from multiple samples). Cellular expression of immune regulatory TGF-beta propeptide (latency-associated peptide) was also increased on Foxp3+ CD4 regulatory T cells (Tregs) of STAT6VT+ mice, as shown by flow cytometry (MFI VT+: 517±200 vs. MFI VT−: 119±100; p<0.05). As also demonstrated by flow cytometry, Foxp3+ CD4 Tregs constituted a higher proportion of T cells from STAT6VT+ T mice (20±5%) compared to T cells from STAT6VT− mice (8±3%) in the mesenteric lymph nodes (p<0.001), and unlike splenic T cells, which displayed similar Foxp3+ CD4 Treg percentages (VT+: 12±2% vs. VT−: 12±3; p:NS).

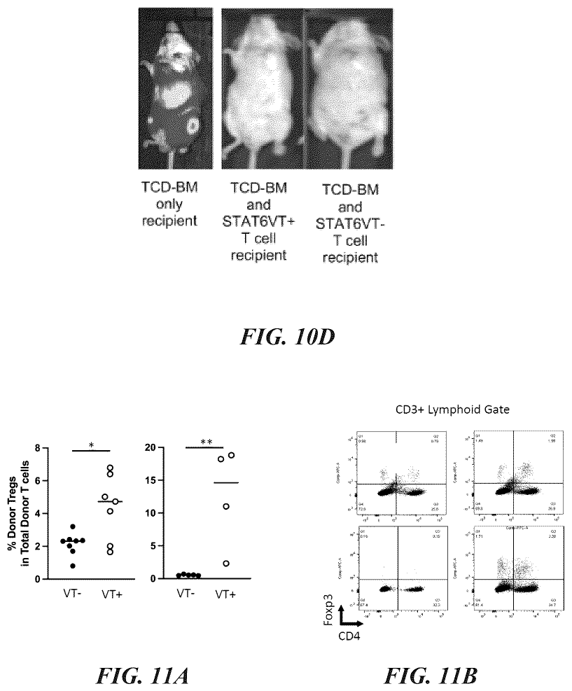

Most CD3+ T cells from STAT6VT+ mice exhibited an effector memory phenotype, with CD62L-CD44+ cells constituting ˜75% of T lymphocyte pool, compared to ˜25% of wildtype C57BL/6 or STAT6VT− T cells (p<0.05). Strikingly, STAT6VT+ transgenic splenic T lymphocytes functioned as effector memory cells after BMT in that they did not cause GVHD and promoted survival of BMT recipients (FIGS. 10A-10C), but still preserved GVT reactivity (FIG. 10D): Tumor-related mortality in BALB/c recipients (H2d) of donor wildtype (WT) C57BL/6 T cell-depleted bone marrow (TCD-BM) cells was 100% compared to 0% tumor-related mortality of BALB/c recipients of donor WT C57BL/6 TCD-BM and STAT6VT+ splenic T cells (p<0.05). Furthermore, and unlike memory T cells, STAT6VT+ T lymphocytes regulated GVHD when co-transferred with GVHD-causing WT splenic C57BL6 T cells and yet maintained the GVT activity: Mice receiving STAT6VT+ T cells alongside WT splenic T cells displayed a phenotype of regulated GVHD with no GVHD- or tumor-related mortality (100% survival), compared to 100% GVHD-related mortality for mice receiving only WT C57BL/6 T cells with WT C57BL/6 TCD-BM cells (p<0.05). This latter group did not show any sign of A20 tumor load by bioluminescence imaging. BALB/c recipients of C57BL/6 WT TCD-BM donor cells were used as another control group, which—as expected—did not display signs of GVHD but all of them died with high A20 tumor load, as shown by bioluminescence imaging (0% GVHD-related mortality but 100% tumor-related mortality). These data suggest that donor T cells expressing a constitutively active STAT6 protein do not only preserve GVT without causing GVHD (similar to donor memory T cells), but STAT6VT+ donor T cells also regulate GVHD and preserve GVT.

Taken together, the data indicate a regulatory role of donor T cell STAT6 expression in the context of memory T cell- and Th2-dependent regulation of GVHD/GVT and suggest that engineering donor T cells to express active STAT6 can have a similar effect, improving the outcome of BMT.

### Example 2

Stimulation of T helper-2 (Th2) pathway regulates acute graft-versus-host disease (GVHD) and preserves the graft-versus-tumor (GVT) effect after bone marrow transplantation (BMT)

Cellular proteins associated with Th2 maturation include the transcription factor STAT6, which promotes immune regulation and alleviates GVHD in different models of BMT. It was investigated whether augmentation of the Th2 pathway in donor T cells by genetic overexpression of STAT6 regulates GVHD and preserves GVT activity. Donor T cells were used from STAT6VT+ transgenic mice on the H2b background whose T cells overexpress a constitutively active form of STAT6 (with a VT/AA mutation at amino acids 625 and 626), driven by the CD2 promoter.

T cells from STAT6VT+ transgenic mice generated high quantities of Th2 and immune regulatory cytokines, such as interleukin-4 (VT+: 2.8±0.2 ng/ml vs. VT−: 0.1±0.03 ng/ml; p<0.0001) or interleukin-10 (VT+: 941±206 pg/ml vs. VT−: 93±13 pg/ml; p<0.0001). Cellular expression of immune regulatory TGFb propeptide (latency-associated peptide) was also increased on Foxp3+ CD4 regulatory T cells (Tregs) of STAT6VT+ mice (MFI VT+: 517±200 vs. MFI VT−: 119±100; p<0.05). Foxp3+ Tregs constituted a higher proportion of T cells from STAT6VT+ T mice (20±5%) compared to T cells from STAT6VT− mice (8±3%) in the mesenteric lymph nodes (p<0.001), unlike splenic T cells, which displayed similar Foxp3+ CD4 Treg percentages (VT+: 12±2% vs. VT−: 12±3; p:NS). Most T cells from STAT6VT+ mice exhibited an effector memory phenotype, with CD62L-CD44+ cells constituting ˜75% of T lymphocyte pool, compared to ˜25% of wildtype C57BL/6 or STAT6VT− T cells (p<0.05).

Strikingly, STAT6VT+ transgenic splenic T lymphocytes functioned as effector memory cells after BMT in that they did not cause GVHD and promoted survival of BMT recipients (H2b®H2d major mismatch model), but still preserved GVT reactivity. Furthermore, and unlike memory T cells, STAT6VT+ T lymphocytes regulated GVHD when cotransferred with GVHD-causing WT splenic T cells and still preserved the GVT activity. Taken together, the data indicate a crucial regulatory role of donor T cell STAT6 expression in the context of memory T cell- and Th2-dependent regulation of GVHD/GVT and suggest that engineering donor T cells to express active STAT6 can have a similar effect, improving the outcome of BMT.

### Conclusions

Thus, stimulation of T helper-2 (Th2) pathway regulates acute graft-versus-host disease (GVHD) and preserves the graft-versus-tumor (GVT) effect after bone marrow transplantation (BMT). Cellular proteins associated with Th2 maturation include the transcription factor STAT6. Genetic overexpression of a constitutively active STAT6 (STAT6VT) in donor T cells regulates GVHD and preserves the GVT.

In particular, continuous STAT6 activity (STAT6VT) in donor T cells promotes Th2 cytokine production. STAT6VT promotes memory T cell phenotype, and does not cause GVHD but preserves the GVT. Moreover, addback of naïve T cells optimizes GVT by STAT6VT+ T cells without causing GVHD Naive T cells cause severe GVHD but have very potent GVT activity. Adding naive T cells to memory T cells allows for an enhanced GVT response and does not cause GVHD. Naive T cell to memory T cell add back can also be performed in the form delayed lymphocyte infusion (DLI).

### Example 3

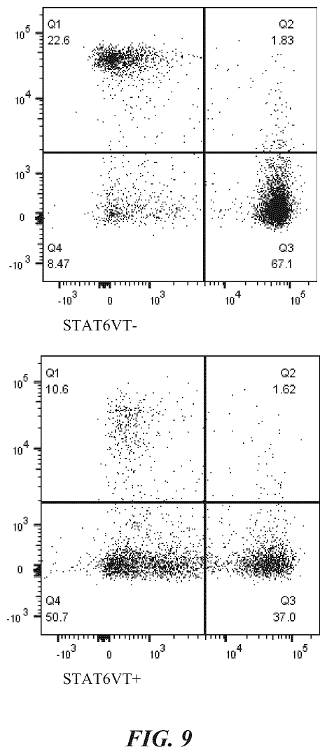

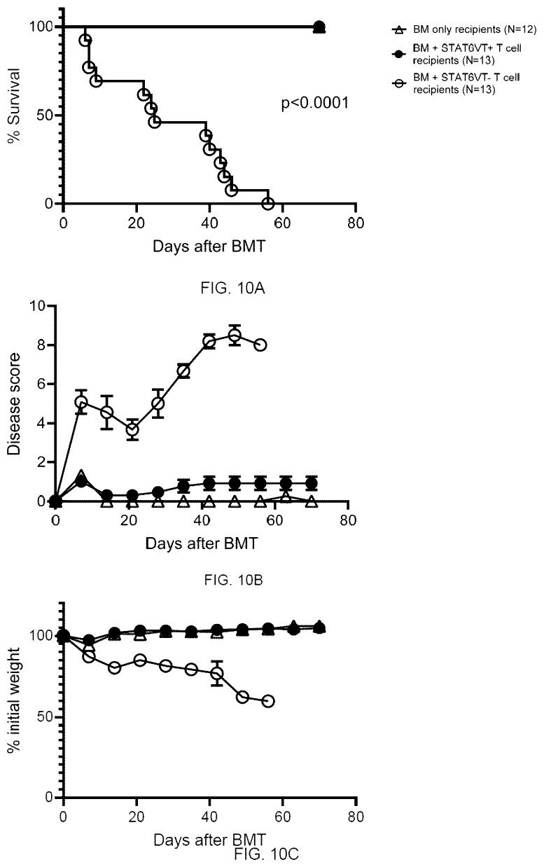

Double negative T cells regulate GVHD. As shown in FIG. 9, an increase in CD3-positive/CD4-negative/CD4-negative (in other words double negative T cells) was observed in STAT6VT+ mice because the left lower quadrant (these are the double negative cells) has more cells in STAT6VT+ mice compared to STAT6VT negative mice.

### Example 4

Add-back of Foxp3+ donor regulatory T cells (Treg), in vitro propagation of Tregs or inducing their in vivo expansion in hematopoietic cell transplant (HCT) recipients offers an approach to treat graft-versus-host disease (GVHD) (PMID: 34575843). Previous studies have shown that helminthic regulation of GVHD in mice is associated with T helper-2 (Th2) and TGF-beta-dependent expansion of donor Tregs (PMID: 37294277, PMID: 30291167, PMID: 30266770, PMID: 25527786). Provided herein is evidence that the expression of STAT6VT in donor T cells was sufficient to promote the expansion of donor Tregs in bone marrow transplant (BMT) recipient mice (FIG. 11; STAT6-mediated expansion of regulatory T cells (Tregs) after bone marrow transplantation).

### Example 5

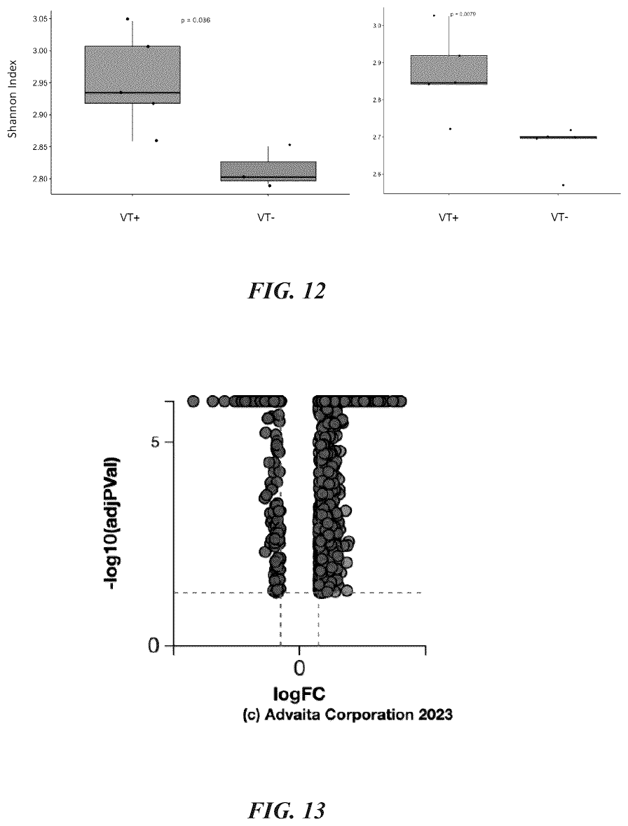

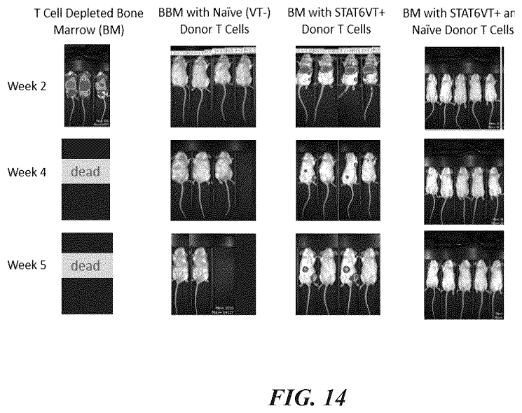

The composition of gut microbiota changes after HCT or BMT. Major changes such as loss of strain diversity occurs of GVHD complicates the transplant procedure. Studies have indicated that the composition of gut microbiota can serve as a biomarker of HCT outcome such as predisposition or occurrence of GVHD, cancer relapse or infectious complications (PMID: 28408426; PMID: 29449660; PMID: 33388483). Provided herein are data demonstrating that transfer of STAT6VT+ donor T cells preserves the diversity of gut microbiota in cecum which can help improve the outcome of transplantation procedure (FIG. 12; Preservation of microbiota diversity in cecum).

### Example 6

Gene or mRNA expression profile of STAT6VT+ T cells differs significantly from STAT6VT− control splenic T cells (FIG. 13; STAT6VT+ splenic T cells are enriched for genes that drive T helper-2 pathway and antitumor reactivity). Genes that are significantly over-expressed compared to STAT6− wild-type T cells include perforin, granzyme A and granzyme B, which are associated with anti-tumor or graft-versus-tumor response after HCT/BMT (PMID: 21856864; PMID: 37611995).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A method to enhance the efficacy of a bone marrow transplant (BMT) in a mammal comprising administering to a mammal in need thereof an amount of a population of hematopoietic stem cells (HSCs) and an amount of isolated T cells that express constitutively active STAT6.

2. The method of claim 1, wherein the hematopoietic stem cells are allogeneic or autologous.

3. The method of claim 1, wherein the T cells are allogeneic or autologous.

4. The method of claim 1, wherein the STAT6 is a human STAT6.

5. The method of claim 1, wherein the immune system of the mammal prior to administering is inactivated.

6. The method of claim 1, wherein the mammal has cancer.

7. The method of claim 6, wherein the cancer is leukemia, myeloma or lymphoma.

8. The method of claim 1, wherein the T cells comprise a viral vector expressing the STAT6.

9. A method to prevent, inhibit or treat an autoimmune disease or to enhance memory cell generation in a mammal comprising administering to a mammal in need thereof an amount of isolated T cells that express constitutively active STAT6, a vector that expresses constitutively active STAT6 or isolated constitutively active STAT6.

10. The method of claim 9, wherein the T cells are allogeneic or autologous.

11. The method of claim 9, wherein the STAT6 is a human STAT6.

12. A method to enhance the immune response to a pathogen in a mammal comprising administering to a mammal in need thereof an amount of isolated T cells that express constitutively active STAT6, a vector that expresses constitutively active STAT6 or isolated constitutively active STAT6 and an immunogen of the pathogen.

13. The method of claim 12, wherein the pathogen is a virus or a bacterium or a coronavirus.

14. The method of claim 1, wherein the STAT6 has at least 86% amino acid sequence identity to SEQ ID NO: 1 or SEQ ID NO: 2.

15. The method of claim 14, wherein the residue at position 548 is alanine, glycine, leucine or isoleucine.

16. A population of isolated human T cells the genome of which comprises a nucleic acid sequence encoding a constitutively active form of human STAT6.
